(19) United States
(12) Patent Application Publication
(10) Pub. No.: US 2024/0122581 A1
(43) Pub. Date: Apr. 18, 2024

(54) SYSTEMS, DEVICES AND METHODS FOR FERTILITY ANALYSIS USING VOICE

(71) Applicant: KVI Brave Fund I Inc., Toronto (CA)

(72) Inventors: Jaycee Morgan Kaufman, Toronto (CA); Yan Fossat, Toronto (CA)

(21) Appl. No.: 18/244,613

(22) Filed: Sep. 11, 2023

Related U.S. Application Data
(60) Provisional application No. 63/416,819, filed on Oct. 17, 2022.

(51) Int. Cl. A61B 10/00; G10L 15/02; G10L 15/22; G16H 50/20

(52) U.S. Cl. CPC A61B 10/0012; G10L 15/02; G10L 15/22; G16H 50/20

(57) ABSTRACT

Provided are methods, systems, devices, and computer readable media for determining fertility level indicators or an ovulation status for a subject. This includes receiving, at a processor in communication with a memory, a voice sample from the subject; extracting, at the processor, at least one voice feature value from the voice sample for at least one predetermined voice feature; determining, at the processor, an ovulation status for the subject based on the at least one voice feature value; and outputting, at an output device, (i) a fertility level indicator for the subject based on the ovulation status, and/or (ii) an ovulation status indicator for the subject based on the ovulation status.

### FIELD

The described embodiments relate to systems, devices and methods for providing fertility analysis using a subject's voice including systems, devices and methods for providing fertility notifications using a subject's voice, and more specifically to systems, devices and methods for providing fertility analysis including an ovulation status indicator or a fertility level indicator based on voice samples including systems, devices and methods for providing fertility analysis including an ovulation status indicator notification or a fertility level indicator notification based on voice samples.

### BACKGROUND

The following is not an admission that anything discussed below is part of the prior art or part of the common general knowledge of a person skilled in the art.

The menstrual cycle is broken up into two phases: the follicular phase and the luteal phase. The follicular phase begins with the first day of menstruation. In a non-pregnant individual, the uterus sheds its lining. Menstruation occurs on average for 3-7 days. After menstruation, estrogen levels begin to rise. When estrogen levels are sufficiently high, individuals produce follicle stimulating hormone (FSH) and luteinizing hormone (LH), both of which prepare the egg for release from the ovary. LH negatively inhibits estrogen production, so estrogen levels begin to decline. When LH peaks, ovulation occurs, indicating the end of the follicular phase. The luteal phase begins with ovulation. After ovulation, progesterone levels increase. LH levels begin to decrease, and estrogen levels increase again. After (average) 12.4 days, both hormones decrease and menstruation begins, indicating the end of the luteal phase and the beginning of the follicular phase.

The fertile window is the 5 days prior to ovulation and the day after and indicates the window in which an individual may become pregnant. The 5 days prior to ovulation comes from the lifespan of sperm, and the one day after comes from the lifespan of the egg.

Conventional solutions for fertility tracking include period-tracking applications for subjects which enables subjects to roughly forecast days during their menstrual cycle that have a higher likelihood of conception. These period-tracking applications however lack accuracy and precision, and do not have capabilities for predicting fertility based on a reliable biomarker of a subject. They do not account for the individual variations in menstrual cycles that may exist.

Existing research has found some correlation with ovulation or menstrual cycle phase with voice features such as the fundamental frequency (f0), shimmer, and jitter (Shoup-Knox, Melanie L., et al. 2019, Pavela Banai, Irena., 2017, Fischer, Julia, et al., 2011). Voice feature change is primarily due to changes in estrogen and progesterone (Zamponi, Virginia, et al., 2021).

These conventional solutions provide for retrospective determinations of correlation between hormone levels and fertility only, i.e., they provide for determining a correlation after-the-fact. Furthermore, because the determinations are made retrospectively, they do not provide any means for providing a useful prediction for a user who wishes to either become pregnant or avoid becoming pregnant, nor do they identify a fertility indicator such as a fertility window. There remains a need for systems and methods for non-invasive fertility predictions for a subject. The fertility predictions may aid a subject in becoming pregnant, or alternatively, may aid a subject in avoiding pregnancy.

Human voice is composed of complex signals that are tightly associated with physiological changes in body systems. Due to the depth of signals that can be analyzed, as well as the wide range of potential physiological dysfunction that manifest in voice signals, voice has quickly gained traction in healthcare and medical research. For example, it has been shown that thyroid hormone imbalance caused the hoarseness of voice, and affected larynx development (Hari Kumar et al., 2016). Unstable pitch and loudness were observed in patients with multiple sclerosis (Noffs et al., 2018). Other recent studies also demonstrated distinct voice characteristics that were associated with various pathological, neurological, and psychiatric disorders, such as congestive heart failure (Maor et al., 2020), Parkinson's disease (Vaicuknyas et al., 2017), Alzheimer's disease (Fraser et al., 2015), post-traumatic stress disorder (Marmar et al., 2019), and autism spectrum disorder (Bonneh et al., 2011). The human voice is now considered as an emerging biomarker, which is inherently non-invasive, low-cost, accessible, and easy to monitor for health conditions in various real-life settings.

Voice signal analysis is an emerging non-invasive technique to examine health conditions. The analysis of human voice data (including voice signal analysis) presents a technical computer-based problem which involves digital signal processing of the voice data. Analysis, including the use of predictive models, requires significant processing capabilities in order to determine biomarker signals and extract relevant information. The sheer number of available biomarker signals poses a challenge since the biomarkers must be efficiently selected in order to reduce processing overhead. Another challenge for voice signal analysis systems performing prediction is that they preferably function in real-time with the voice data collection and on a variety of different processing platforms and operate efficiently to deliver predictions and results to a user in a timely fashion.

A second problem with conventional fertility tracking applications is the risk of misuse of highly private fertility information, conception information, and contraception information about subjects using such applications for tracking menstrual cycle information using software applications.

These conventional solutions including fertility tracking applications lack privacy protections for subjects. There remains a need for systems and methods for fertility tracking applications for subjects that provide sufficient privacy protections to reduce or prevent the risk of misuse of this highly private fertility information, conception information, and contraception information about subjects.

### SUMMARY

The following summary is provided to introduce the reader to the more detailed discussion to follow. The summary is not intended to limit or define any claimed or as yet unclaimed invention. One or more inventions may reside in any combination or sub-combination of the elements or process steps disclosed in any part of this document including its claims and figures.

Provided are systems, devices and methods for providing a fertility indicator or an ovulation status for a subject and associated embodiments.

In a first aspect, there is provided a computer-implemented method for providing a fertility indicator or an ovulation status for a subject, the method comprising: receiving, at a processor in communication with a memory, a voice sample from the subject; extracting, at the processor, at least one voice feature value from the voice sample for at least one predetermined voice feature; determining, at the processor, an ovulation status for the subject based on the at least one voice feature value; and outputting, at an output device, (i) a fertility level indicator for the subject based on the ovulation status, and or (ii) an ovulation status indicator for the subject based on the ovulation status.

In one or more embodiments, the fertility level indicator for the subject may comprise a historical fertility indicator for the subject, optionally wherein the historical fertility indicator may be provided over a single menstrual cycle of the subject.

In one or more embodiments, the fertility level indicator for the subject may be a category comprising fertile or not fertile.

In one or more embodiments, the fertility level indicator for the subject may be a category comprising: menstruating, follicular, or luteal.

In one or more embodiments, the fertility level indicator for the subject may be a category comprising: a low category, a medium category, and a high category.

In one or more embodiments, the low category, the medium category, and the high category may each comprise predetermined thresholds.

In one or more embodiments, the ovulation status indicator may comprise an indicator of ovulation based on a transition from the follicular category to the luteal category.

In one or more embodiments, the fertility level indicator may comprise a percentage.

In one or more embodiments, the at least one predetermined voice feature may be at least one selected from a group of a fundamental frequency (F0) feature, a spectral flux feature, a jitter feature, a harmonic to noise ratio feature, a shimmer feature, and an alpha ratio feature.

In one or more embodiments, the at least one predetermined voice feature may comprise a fundamental frequency standard deviation feature, and wherein the determining, at the processor, the ovulation status for the subject may comprise: determining, at the processor, the at least one voice feature value comprising a mean fundamental frequency standard deviation of the voice sample and a deviation of the fundamental frequency standard deviation of the voice sample from the mean fundamental frequency standard deviation of the voice sample; and when the deviation is greater than a predetermined threshold, determine an occurrence of ovulation.

In one or more embodiments, the predetermined threshold may be 20%.

In one or more embodiments, the determining, at the processor, the ovulation status for the subject may comprise: determining, at the processor, the at least one voice feature value comprising a derivative of the fundamental frequency (F0) feature; and determining the ovulation status based on the derivative of the fundamental frequency (F0) feature.

In one or more embodiments, the at least one predetermined voice feature may comprise a non-patient specific feature.

In one or more embodiments, the ovulation status may be determined based on a negative derivative of the fundamental frequency (F0).

In one or more embodiments, the at least one predetermined voice feature may be a shimmer mean feature and wherein the determining the ovulation status for the subject may comprise: determining the at least one voice feature value comprising at least one local maximum shimmer mean feature value of the voice sample, and determining the ovulation status based on the at least one local maximum shimmer mean feature value.

In one or more embodiments, the ovulation status may be determined based on a decision tree, the decision tree using the at least one predetermined voice feature of the voice sample to determine the ovulation status.

In one or more embodiments, the method may further comprise: receiving, at the processor from a user input device, onboarding information comprising an age of the subject, a first day of a menstrual cycle of the subject, a menstrual status of the subject, and optionally a birth control status of the subject; and wherein the method further comprises determining, at the processor, the ovulation status for the subject based on at least one selected from a group of the age of the subject, the first day of a menstrual cycle of the subject, the menstrual status of the subject, and optionally on the birth control status of the subject.

In one or more embodiments, the onboarding information may comprise an initial voice sample from the subject, the initial voice sample may indicate the subject's consent to the determining and outputting the ovulation status.

In one or more embodiments, the method may further comprise: authenticating the subject by comparing the voice sample to the initial voice sample prior to performing the determining and outputting of the ovulation status.

In one or more embodiments, the method may further comprise deleting the voice sample.

In one or more embodiments, the fertility level indicator for the subject may comprise a timeline interface for the subject; and wherein the outputting the fertility level indicator may comprise displaying the timeline interface on a display device.

In one or more embodiments, the timeline interface may comprise a menstruation window, a first non-fertile window, a fertile window, a second non-fertile window, and an indicator of the subject's position along the timeline interface.

In one or more embodiments, the method may further comprise: receiving, at an audio input device, the voice sample; wherein the voice sample may be collected contemporaneously from the subject's speech.

In one or more embodiments, the method may further comprise: receiving, at an audio input device, the voice sample; and wherein the voice sample may comprise a prompt vocalized by the subject, optionally wherein the predetermined phrase comprises a date or a time.

In one or more embodiments, the method may further comprise: displaying, at the display device, a reminder notification to the subject to collect the voice sample.

In one or more embodiments, the reminder notification may be displayed to the subject at a predetermined time of the day.

In one or more embodiments, the reminder notification may comprise the predetermined phrase.

In one or more embodiments, the method may further comprise: providing, at a user device, a conception application for assisting the subject to become pregnant; wherein the voice sample may be obtained at the user device using the conception application.

In one or more embodiments, the method may further comprise generating, at the user device, a conception notification associated with the conception application, wherein the conception notification may comprise the fertility level indicator.

In one or more embodiments, the conception notification may be generated based on a percentage value of the fertility level indicator.

In one or more embodiments, the method may further comprise providing, at a user device, a contraception application for assisting the subject to avoid becoming pregnant.

In one or more embodiments, the method may further comprise: generating, at the user device, a contraception notification associated with the contraception application, the contraception notification comprising the fertility level indicator.

In one or more embodiments, the contraception notification may be generated based on a percentage value of the fertility level indicator.

In one or more embodiments, the user device may be used by the subject.

In one or more embodiments, the user device may be used by a clinician.

In a second aspect, there is provided a system for determining a fertility level for a subject, the system comprising: a memory; a processor in communication with the memory, the processor configured to operate the method of any one of the embodiments herein.

In a third aspect, there is provided a device for determining a fertility level for a subject, the device comprising: a memory; a processor in communication with the memory, the processor configured to operate the method of any one of the embodiments herein.

In a fourth aspect, there is provided a computer program product for determining a fertility level for a subject, the device comprising: a memory; a processor in communication with the memory, the processor configured to operate the method of any one of the embodiments herein.

It will be appreciated by a person skilled in the art that a system, device, method or computer program product disclosed herein may embody any one or more of the features contained herein and that the features may be used in any particular combination or sub-combination. Other features and advantages of the present application will become apparent from the following detailed description taken together with the accompanying drawings. It should be understood, however, that the detailed description and the specific examples are given by way of illustration only, since various changes and modifications within the scope of the application will become apparent to those skilled in the art from this detailed description.

### BRIEF DESCRIPTION OF THE DIAGRAMS

For a better understanding of the various examples described herein, and to show more clearly how these various examples may be carried into effect, reference will be made, by way of example, to the accompanying drawings which show at least one example, and which are now described. The drawings are not intended to limit the scope of the teachings described herein.

A preferred embodiment of the present invention will now be described in detail with reference to the diagrams, in which:

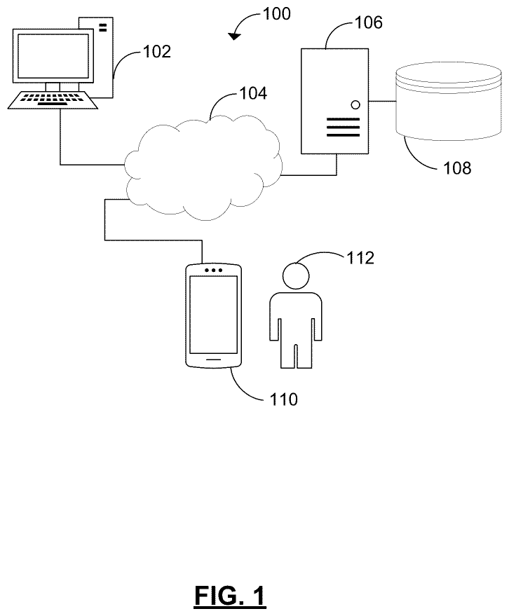

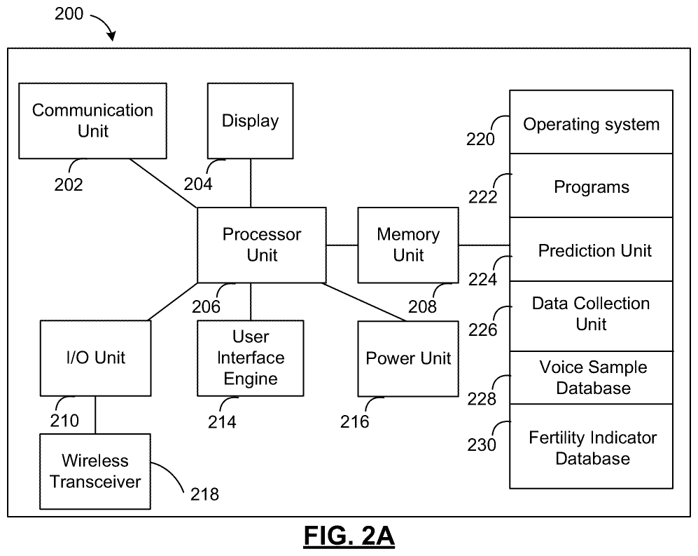

FIG. 1 shows a system diagram in accordance with one or more embodiments.

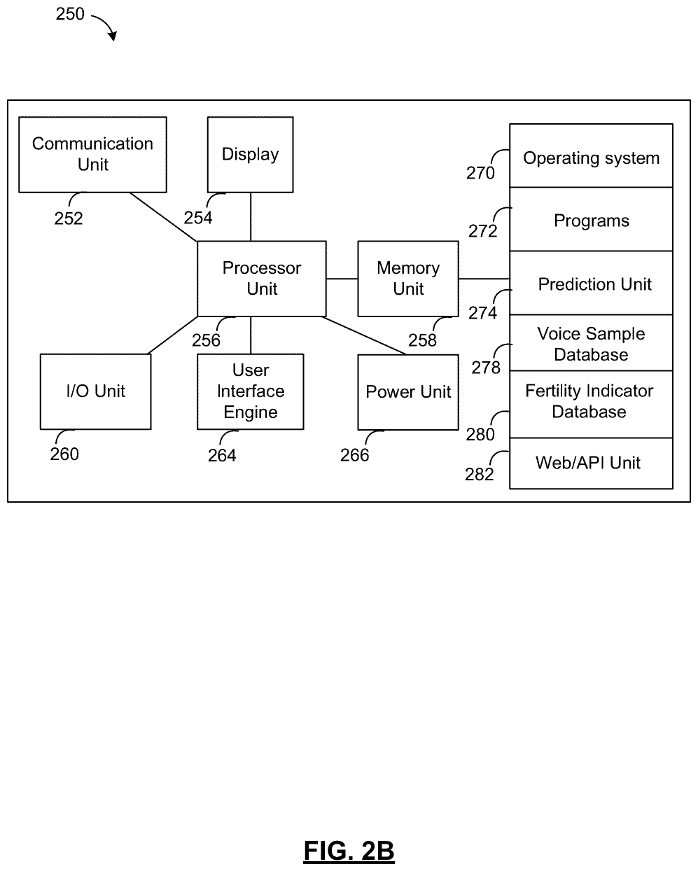

FIG. 2A shows a device diagram in accordance with one or more embodiments.

FIG. 2B shows a device diagram in accordance with one or more embodiments.

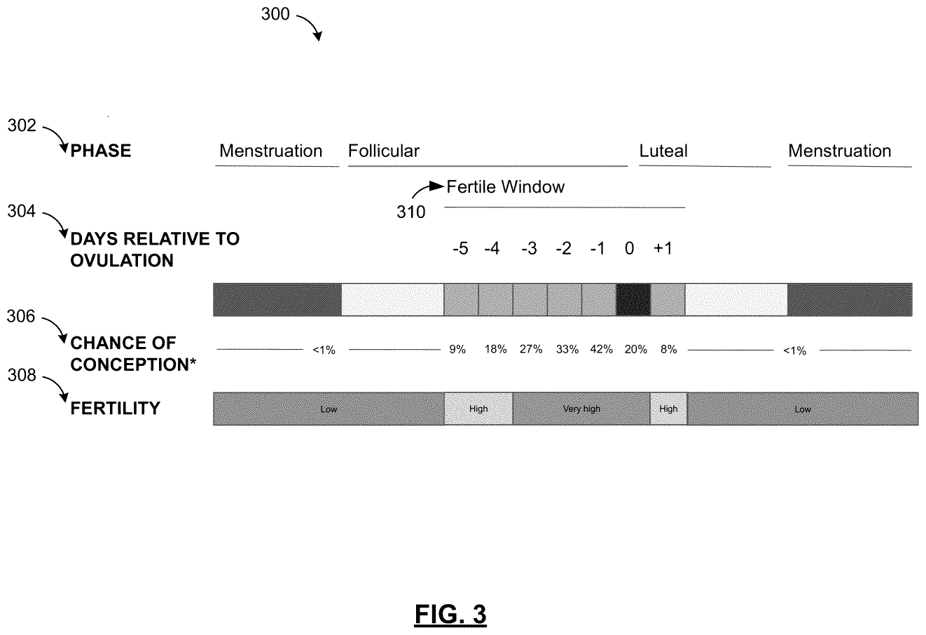

FIG. 3 shows a menstrual cycle diagram of a subject in accordance with one or more embodiments.

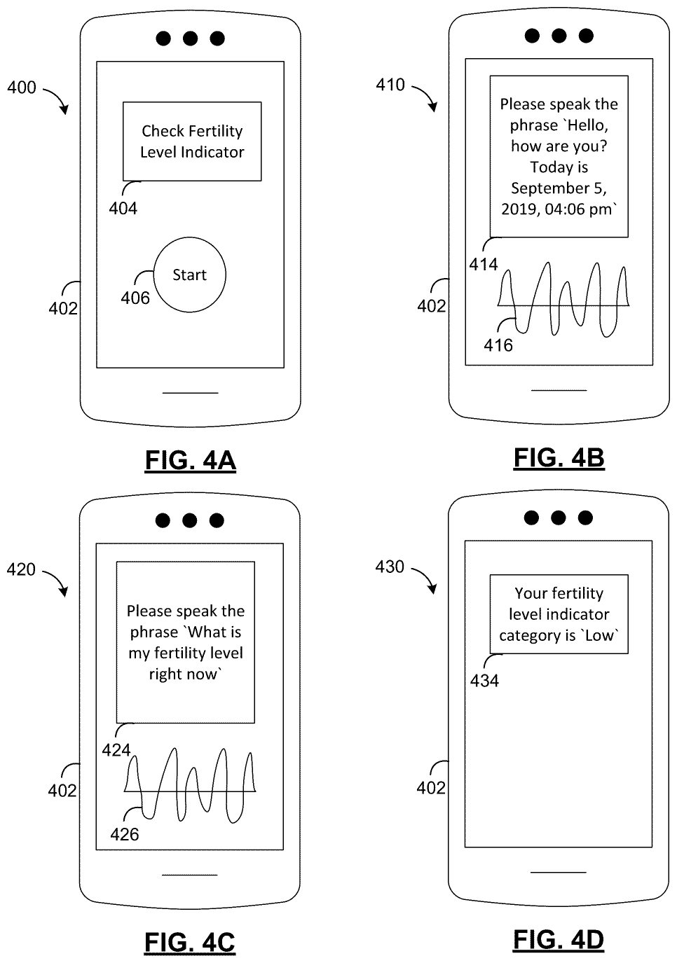

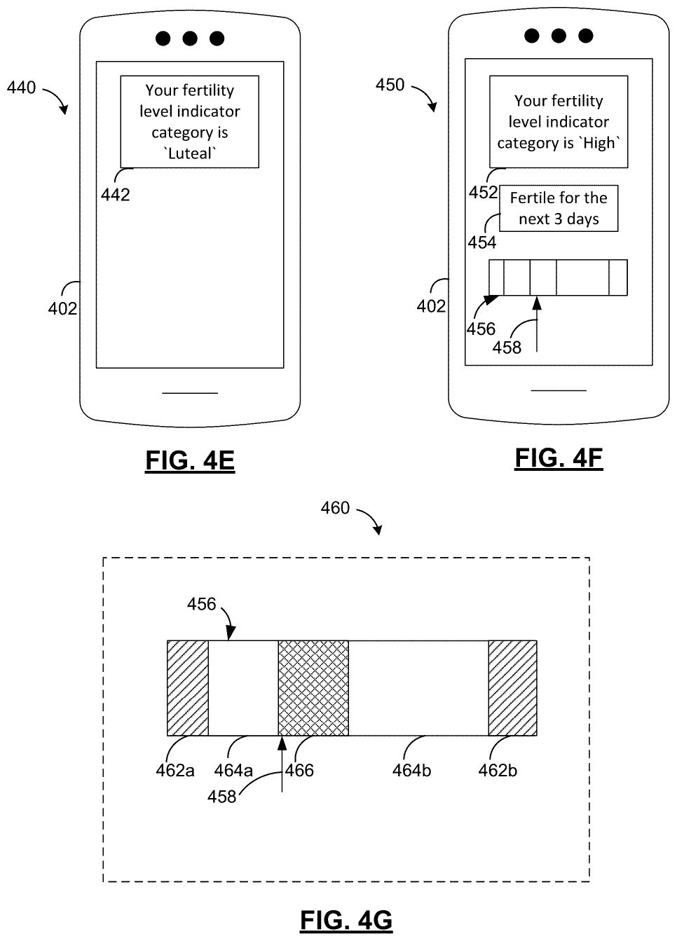

FIGS. 4A, 4B, 4C, 4D, 4E, 4F, and 4G show user interface diagrams in accordance with one or more embodiments.

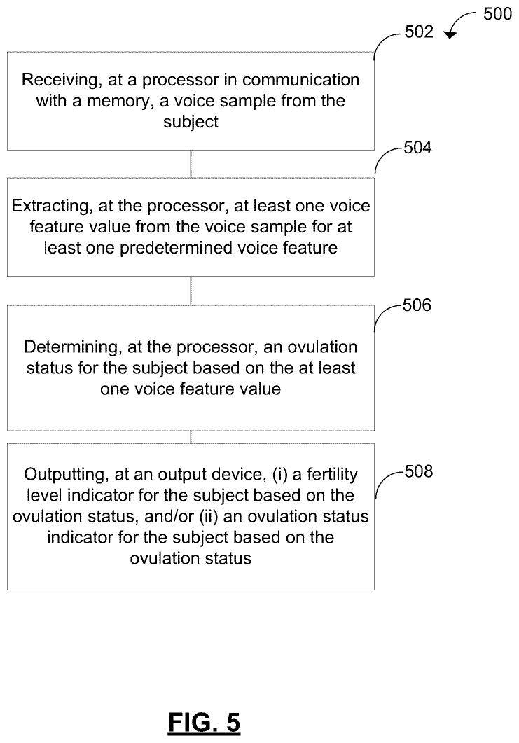

FIG. 5 shows a computer-implemented method diagram in accordance with one or more embodiments.

FIG. 6 shows another computer implemented method diagram in accordance with one or more embodiments.

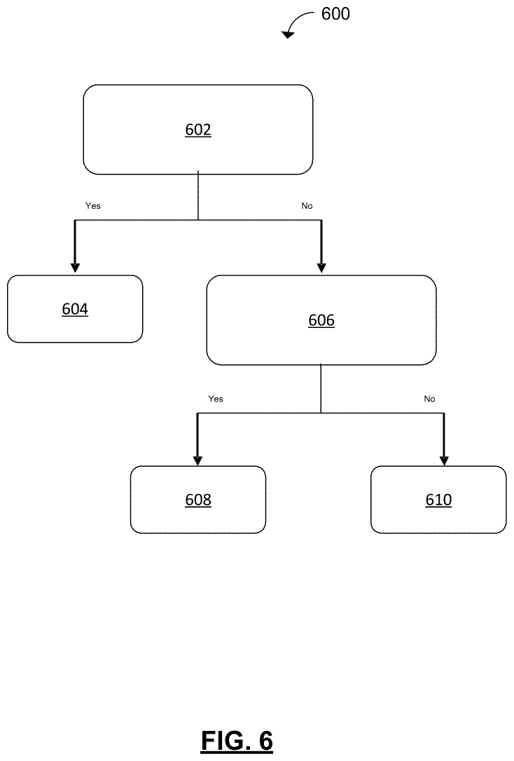

FIG. 7 shows a feature value in accordance with one or more embodiments.

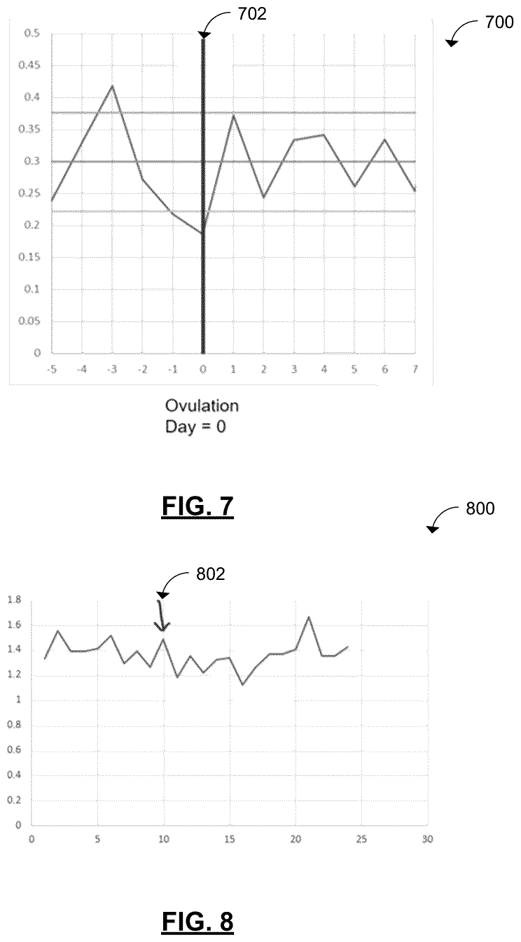

FIG. 8 shows another feature value in accordance with one or more embodiments.

### DESCRIPTION OF EXEMPLARY EMBODIMENTS

Various apparatuses or methods will be described below to provide an example of the claimed subject matter. No example described below limits any claimed subject matter and any claimed subject matter may cover methods or apparatuses that differ from those described below. The claimed subject matter is not limited to apparatuses or methods having all of the features of any one apparatus or method described below or to features common to multiple or all of the apparatuses or methods described below. It is possible that an apparatus or methods described below is not an example that is recited in any claimed subject matter. Any subject matter disclosed in an apparatus or methods described below that is not claimed in this document may be the subject matter of another protective instrument, for example, a continuing patent application, and the applicants, inventors or owners do not intend to abandon, disclaim or dedicate to the public any such invention by its disclosure in this document.

Furthermore, it will be appreciated that for simplicity and clarity of illustration, where considered appropriate, reference numerals may be repeated among the figures to indicate corresponding or analogous elements. In addition, numerous specific details are set forth in order to provide a thorough understanding of the examples described herein. However, it will be understood by those of ordinary skill in the art that the examples described herein may be practiced without these specific details. In other instances, well-known methods, procedures and components have not been described in detail so as not to obscure the examples described herein. Also, the description is not to be considered as limiting the scope of the examples described herein.

It should also be noted that the terms “coupled” or “coupling” as used herein can have several different meanings depending in the context in which these terms are used. For example, the terms coupled or coupling can have a mechanical, electrical or communicative connotation. For example, as used herein, the terms coupled or coupling can indicate that two elements or devices can be directly connected to one another or connected to one another through one or more intermediate elements or devices via an electrical element, electrical signal or a mechanical element depending on the particular context. Furthermore, the term “communicative coupling” indicates that an element or device can electrically, optically, or wirelessly send data to another element or device as well as receive data from another element or device.

It should also be noted that, as used herein, the wording “and/or” is intended to represent an inclusive-or. That is, “X and/or Y” is intended to mean X or Y or both, for example. As a further example, “X, Y, and/or Z” is intended to mean X or Y or Z or any combination thereof.

It should be noted that terms of degree such as “substantially”, “about” and “approximately” as used herein mean a reasonable amount of deviation of the modified term such that the end result is not significantly changed. These terms of degree may also be construed as including a deviation of the modified term if this deviation would not negate the meaning of the term it modifies.

Furthermore, the recitation of numerical ranges by endpoints herein includes all numbers and fractions subsumed within that range (e.g. 1 to 5 includes 1, 1.5, 2, 2.75, 3, 3.90, 4, and 5). It is also to be understood that all numbers and fractions thereof are presumed to be modified by the term “about” which means a variation of up to a certain amount of the number to which reference is being made if the end result is not significantly changed.

Some elements herein may be identified by a part number, which is composed of a base number followed by an alphabetical or subscript-numerical suffix (e.g. 112a, or 1121). Multiple elements herein may be identified by part numbers that share a base number in common and that differ by their suffixes (e.g. 1121, 1122, and 1123). All elements with a common base number may be referred to collectively or generically using the base number without a suffix (e.g. 112).

The example systems and methods described herein may be implemented in hardware or software, or a combination of both. In some cases, the examples described herein may be implemented, at least in part, by using one or more computer programs, executing on one or more programmable devices comprising at least one processing element, a data storage element (including volatile and non-volatile memory and/or storage elements), and at least one communication interface. These devices may also have at least one input device (e.g. a keyboard, mouse, a touchscreen, and the like), and at least one output device (e.g. a display screen, a printer, a wireless radio, and the like) depending on the nature of the device. For example and without limitation, the programmable devices (referred to below as computing devices) may be a server, network appliance, embedded device, computer expansion module, a personal computer, laptop, personal data assistant, cellular telephone, smart-phone device, tablet computer, a wireless device or any other computing device capable of being configured to carry out the methods described herein.

In some examples, the communication interface may be a network communication interface. In examples in which elements are combined, the communication interface may be a software communication interface, such as those for inter-process communication (IPC). In still other examples, there may be a combination of communication interfaces implemented as hardware, software, and a combination thereof.

Program code may be applied to input data to perform the functions described herein and to generate output information. The output information is applied to one or more output devices, in known fashion.

Each program may be implemented in a high-level procedural, declarative, functional or object oriented programming and/or scripting language, or both, to communicate with a computer system. However, the programs may be implemented in assembly or machine language, if desired. In any case, the language may be a compiled or interpreted language. Each such computer program may be stored on a storage media or a device (e.g. ROM, magnetic disk, optical disc) readable by a general or special purpose programmable computer, for configuring and operating the computer when the storage media or device is read by the computer to perform the procedures described herein. Examples of the system may also be considered to be implemented as a non-transitory computer-readable storage medium, configured with a computer program, where the storage medium so configured causes a computer to operate in a specific and predefined manner to perform the functions described herein.

Furthermore, the example system, processes and methods are capable of being distributed in a computer program product comprising a computer readable medium that bears computer usable instructions for one or more processors. The medium may be provided in various forms, including one or more diskettes, compact disks, tapes, chips, wireline transmissions, satellite transmissions, internet transmission or downloads, magnetic and electronic storage media, digital and analog signals, and the like. The computer useable instructions may also be in various forms, including compiled and non-compiled code.

Various examples of systems, methods and computer programs products are described herein. Modifications and variations may be made to these examples without departing from the scope of the invention, which is limited only by the appended claims. Also, in the various user interfaces illustrated in the figures, it will be understood that the illustrated user interface text and controls are provided as examples only and are not meant to be limiting. Other suitable user interface elements may be used with alternative implementations of the systems and methods described herein.

As used herein, the term “user” refers to a user of a user device, and the term “subject” refers to a subject whose measurements are being collected. The user and the subject may be the same person, or they may be different persons in the case where one individual operates the user device and another individual is the subject. For example, in one embodiment the user may be a health care professional such as a nurse or doctor and the subject is a human patient.

Reference is first made to FIG. 1, which shows a system diagram 100 of a computer-implemented prediction system for determining a fertility indicator or an ovulation status for a subject. The fertility indicator or an ovulation status prediction system may include one or more computer devices 102, a network 104, one or more servers 106, one or more data stores 108, and one or more user devices 110 for one or more users 112.

The computer-implemented prediction system performs voice analysis which may be used to predict and identify a fertility indicator or an ovulation status for a subject corresponding to the subject's fertile window for the purposes of conception or contraception. The prediction system may identify the day of ovulation in a fertile subject by analysis of a voice recording made by the subject. Optionally, the voice recording may be made at a regular interval, for example, daily.

The daily interval recordings may be collected generally at the same time every day. A software application running on the user device 110 may provide one or more notifications, including notifications related to conception and contraception for a user 112 of the user device 110 (including where the user is the subject of the measurements). The notifications may remind the user to collect voice samples from the subject (themselves, in the case where the user and the subject are the same individual).

The one or more computer devices 102 may be used by a clinician user such as an administrator, clinician, fertility clinician, or other medical professional to access a software application (not shown) running on server 106 over network 104. In one embodiment, the one or more computer devices 102 may access a web application hosted at server 106 using a browser for reviewing fertility indicator or ovulation status predictions given to the users 112 (including users who are subjects) using user devices 110.

The one or more user devices 110 may download an application (including downloading from an App Store such as the Apple® App Store or the Google® Play Store) for determining fertility indicator or ovulation status predictions for the users 112 (including subjects who are users) using user devices 116.

The one or more user devices 110 may be any two-way communication device with capabilities to communicate with other devices. A user device 110 may be a mobile device such as mobile devices running the Google® Android® operating system or Apple® iOS® operating system. A user device 110 may be a smart speaker, such as an Amazon® Alexa® device, or a Google® Home® device. A user device 110 may be a smart watch such as the Apple® Watch, Samsung® Galaxy® watch, a Fitbit® device, or others as known. A user device 110 may be a purpose-built sensor system attached to the body of, or on the clothing of, a user.

A user device 110 may be the personal device of a user, or may be a device provided by an employer. The one or more user devices 110 may be used by an end user 112 to access the software application (not shown) running on server 106 over network 104. In one embodiment, the one or more user devices 110 may access a web application hosted at server 106 using a browser for determining fertility indicator or ovulation status predictions. In an alternate embodiment, the one or more user devices 110 may download an application (including downloading from an App Store such as the Apple® App Store or the Google® Play Store) for determining fertility indicator or an ovulation status predictions. The user device 110 may be a desktop computer, mobile device, or laptop computer. The user device 110 may be in communication with server 106, and may allow a user 112 to review a user profile stored in a database at data store 108, including historical fertility indicator or an ovulation status predictions. The users 112 using user devices 110 may provide one or more voice samples using a software application, and may receive a fertility indicator or an ovulation status prediction based on the one or more voice samples as described herein.

The one or more user devices 110 may each have one or more audio sensors. The one or more audio sensors may be in an array. The audio sensors may be used by a user 112 of the software application to record a voice sample into the memory of the user device 110. The one or more audio sensors may be an electret microphone onboard the user device, MEMS microphone onboard the user device, a Bluetooth enabled connection to a wireless microphone, a line in, etc.

The software application running on the one or more user devices 110 may communicate with server 106 using an Application Programming Interface (API) endpoint, and may send and receive voice sample data, user data, mobile device data, and mobile device metadata.

The software application running on the one or more user devices 110 may display one or more user interfaces on a display device of the user device, including, but not limited to, the user interfaces shown in FIGS. 4A, 4B, 4C, 4D, 4E, 4F, and 4G.

Network 104 may be any network or network components capable of carrying data including the Internet, Ethernet, fiber optics, satellite, mobile, wireless (e.g. Wi-Fi, WiMAX), SS7 signaling network, fixed line, local area network (LAN), wide area network (WAN), a direct point-to-point connection, mobile data networks (e.g., Universal Mobile Telecommunications System (UMTS), 3GPP Long-Term Evolution Advanced (LTE Advanced), Worldwide Interoperability for Microwave Access (WiMAX), etc.) and others, including any combination of these.

The server 106 is in network communication with the one or more user devices 110 and the one or more computer devices 102. The server 106 may further be in communication with a database at data store 108. The database at data store 108 and the server 106 may be provided on the same server device, may be configured as virtual machines, or may be configured as containers. The server 106 and a database at data store 108 may run on a cloud provider such as Amazon® Web Services (AWS®).

The server 106 may host a web application or an Application Programming Interface (API) endpoint that the one or more user devices 110 may interact with via network 104. The server 106 may make calls to the mobile device 110 to poll for voice sample data. Further, the server 106 may make calls to the database at data store 108 to query subject data, voice sample data, fertility data, or other data received from the users 112 of the one or more user devices 110. The requests made to the API endpoint of server 106 may be made in a variety of different formats, such as JavaScript Object Notation (JSON) or eXtensible Markup Language (XML). The voice sample data may be transmitted between the server 106 and the user device 110 in a variety of different formats, including MP3, MP4, AAC, WAV, Ogg Vorbis, FLAC, or other audio data formats as known. The voice sample data may be stored as Pulse-Code Modulation (PCM) data. The voice sample data may be recorded at 22,050 Hz or 44,100 Hz. The voice sample data may be collected as a mono signal, or a stereo signal. The voice sample data may be encrypted by user device 110 prior to transmission to server 106. The voice sample data may be encrypted du The voice sample data received by the data store 108 from the one or more user devices 110 may be stored in the database at data store 108, or may be stored in a file system at data store 108. The file system may be a redundant storage device at the data store 108, or may be another service such as Amazon® S3, or Dropbox.

The database of data store 108 may store subject information including fertility data, subject and/or user information including subject and/or user profile information, and configuration information. The database of data store 108 may be a Structured Query Language (SQL) such as PostgreSQL or MySQL or a not only SQL (NoSQL) database such as MongoDB.

FIG. 2 shows a user device diagram 200 showing detail of the one or more user devices 110 in FIG. 1.

The user device 200 includes one or more of a communication unit 202, a display 204, a processor unit 206, a memory unit 208, I/O unit 210, a user interface engine 214, a power unit 216, and a wireless transceiver 218. The user device 200 may be a laptop, gaming system, smart speaker device, mobile phone device, smart watch or others as are known. The user device 200 may be a passive sensor system proximate to the user, for example, a device worn on user, or on the clothing of the user.

The communication unit 202 can include wired or wireless connection capabilities. The communication unit 202 can include a radio that communicates utilizing CDMA, GSM, GPRS or Bluetooth protocol according to standards such as IEEE 802.11a, 802.11b, 802.11g, or 802.11n. The communication unit 202 can be used by the mobile device 200 to communicate with other devices or computers.

Communication unit 202 may communicate with the wireless transceiver 218 to transmit and receive information via a local wireless network with a microphone. In an alternate embodiment, the communication unit 202 may communicate with the wireless transceiver 218 to transmit and receive information via local wireless network with an optional handheld device associated with the fertility prediction device 200. The communication unit 202 may provide communications over the local wireless network using a protocol such as Bluetooth (BT) or Bluetooth Low Energy (BLE).

The display 204 may be an LED or LCD based display, and may be a touch sensitive user input device that supports gestures.

The processor unit 206 controls the operation of the mobile device 200. The processor unit 206 can be any suitable processor, controller or digital signal processor that can provide sufficient processing power depending on the configuration, purposes and requirements of the user device 200 as is known by those skilled in the art. For example, the processor unit 206 may be a high performance general processor. In alternative embodiments, the processor unit 206 can include more than one processor with each processor being configured to perform different dedicated tasks. In alternative embodiments, it may be possible to use specialized hardware to provide some of the functions provided by the processor unit 206. For example, the processor unit 206 may include a standard processor, such as an Intel® processor, an ARM® processor or a microcontroller.

The processor unit 206 can also execute a user interface (UI) engine 214 that is used to generate various UIs, some examples of which are shown and described herein, such as interfaces shown in FIGS. 4A-4G.

The present systems, devices and methods may provide an improvement in the operation of the processor unit 206 by ensuring the analysis of voice data and fertility indicator predictions are made using relevant biomarkers. The reduced processing required for the relevant biomarkers in the analysis (as compared with processing the superset of all biomarkers) reduces the processing burden required to make fertility indicator or an ovulation status predictions based on voice data.

The memory unit 208 comprises software code for implementing an operating system 220, programs 222, prediction unit 224, data collection unit 226, voice sample database 228, and fertility indicator database 230.

The present systems and methods may provide an improvement in the operation of the memory unit 208 by ensuring the analysis of voice data is performed using relevant biomarkers and thus only relevant biomarker data is stored. The reduced storage required for the relevant biomarkers in the analysis (as compared with processing the superset of all biomarkers) reduces the memory overhead required to make fertility indicator or an ovulation status predictions based on voice data.

The memory unit 208 can include RAM, ROM, one or more hard drives, one or more flash drives or some other suitable data storage elements such as disk drives, etc. The memory unit 208 is used to store an operating system 220 and programs 222 as is commonly known by those skilled in the art.

The I/O unit 210 can include at least one of a mouse, a keyboard, a touch screen, a thumbwheel, a track-pad, a track-ball, a card-reader, an audio source, a microphone, voice recognition software and the like again depending on the particular implementation of the user device 200. In some cases, some of these components can be integrated with one another.

The user interface engine 214 is configured to generate interfaces for users to configure voice measurement, connect to a fertility monitoring device or audio input device, record voice data, view fertility predictions, view voice sample data, etc. The various interfaces generated by the user interface engine 214 are displayed to the user on display 204.

The power unit 216 can be any suitable power source that provides power to the user device 200 such as a power adaptor or a rechargeable battery pack depending on the implementation of the user device 200 as is known by those skilled in the art.

The operating system 220 may provide various basic operational processes for the user device 200. For example, the operating system 220 may be a mobile operating system such as Google® Android® operating system, or Apple® iOS® operating system, or another operating system.

The programs 222 include various user programs so that a user can interact with the user device 200 to perform various functions such as, but not limited to, viewing fertility indictor data, voice data, recording voice samples, receiving and viewing fertility indicator data or ovulation status data from a device worn on or about the subject, receiving any other data related to fertility predictions, as well as receiving messages, notifications and alarms as the case may be. The programs 422 may be downloaded from an application store “app store”) such as the Apple® App Store® or the Google® Play Store®.

In one or more embodiments, the programs 222 may include a conception application. The conception application may record voice samples from the user and report the subject's fertility indicator or an ovulation status category/level. Such a conception application may integrate with a health tracker of the individual such as a Fitbit®, or Apple® Watch such that additional measurement data may be collected. The conception application may record historical fertility indicator or an ovulation status predictions in order to determine changes in the user's fertility indicator or an ovulation status levels. The embodiments described herein may allow for a user to check their relative fertility level indicator using voice samples, and may allow a user who wishes to conceive to avoid having to track their menstrual cycle manually using a calendar. The conception application may use the fertility indicator or an ovulation status level to generate a notification to a user. The notification may include a mobile notification such as an app notification, a text notification, an email notification, or another notification that is known. The conception application may operate using the method of FIG. 5.

In one or more embodiments, the programs 222 may include a contraception application. The contraception application may record voice samples from the user and report the subject's fertility indicator or an ovulation status category/level. Such a contraception application may integrate with a health tracker of the individual such as a Fitbit®, or Apple® Watch such that additional measurement data may be collected. The contraception application may record historical fertility indicator or an ovulation status predictions in order to determine changes in the user's fertility indicator or an ovulation status levels. The embodiments described herein may allow for a user to check their relative fertility level indicator using voice samples, and may allow a user who wishes to avoid becoming pregnant to avoid having to track their menstrual cycle manually using a calendar. The contraception application may use the fertility indicator or an ovulation status level to generate a notification to a user. The notification may include a mobile notification such as an app notification, a text notification, an email notification, or another notification that is known. The contraception application may operate using the method of FIG. 5.

In one or more embodiments, the programs 222 may include a third-party application. The third-party application may be, for example, a third-party period or menstrual cycle tracking application. The third-party application may record voice samples from the user and report the subject's fertility indicator or an ovulation status category/level. Such a third-party application may communicate via an API with the prediction unit 224. Such a third-party application may integrate with a health tracker of the individual such as a Fitbit®, or Apple® Watch such that additional measurement data may be collected. The third-party application may record historical fertility indicator or an ovulation status predictions in order to determine changes in the user's fertility indicator or an ovulation status levels. The third-party application may use the fertility indicator or an ovulation status level to generate a notification to a user. The notification may include a mobile notification such as an app notification, a text notification, an email notification, or another notification that is known. The third-party application may operate using the method of FIG. 5.

In one or more embodiments, the programs 222 may include a companion application for a hormone detection product, for example, Clearblue®. Alternatively, the companion application may be provided by a fertility clinic to a subject. The companion application may record voice samples from the user and report the subject's fertility indicator or an ovulation status category/level. The companion application may further enable a user to input the result of the hormone detection product. Such a third-party application may integrate with a health tracker of the individual such as a Fitbit®, or Apple® Watch such that additional measurement data may be collected. The third-party application may record historical fertility indicator or an ovulation status predictions in order to determine changes in the user's fertility indicator or an ovulation status levels. This may include historical results from the hormone detection device(s). The companion application may use the fertility indicator or an ovulation status level to generate a notification to a user. The notification may include a mobile notification such as an app notification, a text notification, an email notification, or another notification that is known. The companion application may operate using the method of FIG. 5.

In one or more embodiments, the programs 422 may include a smart speaker application, operable to interact with a user using voice prompts, and receptive of voice commands. In such an embodiment, the voice commands the user provides as input may be used as voice sample data as described herein. In this case, a user may request their fertility indicator or an ovulation status prediction by prompting the smart speaker “Alexa, how is my fertility level?” or similar. The smart speaker application may passively monitor the user's fertility indicator or an ovulation status levels by way of the voice command voice samples, and may alert the user with an audio notification when it changes categories. The smart speaker application may follow the method of FIG. 5.

In one or more embodiments, the programs 422 may include a smart watch application for outputting information including a fertility indicator or an ovulation status level or category on a watch face. The smart watch application may enable a user to provide voice prompts using an input device of the watch and check fertility predictions on an output device of the watch. The smart watch application may follow the method of FIG. 5.

The programs 422 may enable a user to diarize the predictions in a fertility diary request. The fertility diary request may allow a user to document at different times, predictions made using the method of FIG. 5 along with information about fertility treatments or sexual intercourse, or prophylactic use which may impact upon the user's desire to either become pregnant, or to avoid pregnancy.

Based on the fertility indicator or an ovulation status level, and the user's diarized fertility requests, the conception application or the contraception application may generate a change recommendation personalized for the user. The change recommendation including actions, behavior patterns, or other information to assist the user in either becoming pregnant or avoiding pregnancy.

In one or more embodiments, the programs 222 may include a passive fertility application that may receive audio inputs, transmit voice samples to a server, optionally receive fertility indicator or an ovulation status predictions, and optionally provide alerts or notifications to the user's device to the user automatically and without user prompting. In one or more embodiments, the passive sensor application may be connected wirelessly to a user device such as a mobile phone, and may cause an email, text message, or application notification to be displayed to a user on the user device. The passive sensor application may follow the method of FIG. 5.

In one or more embodiments, the passive sensor application may provide a notification to the user such as to use sexual prophylactics such as condoms in order to avoid pregnancy.

In one or more embodiments, the programs 222 may include an educational application. For example, in one embodiment programs 222 include an educational application for helping subjects understand their fertility levels for conception or contraception. The educational program may communicate behavioral changes to the user, and may use the user's voice samples to tailor educational content presented to them on the user device. The educational application may follow the method of FIG. 5.

The prediction unit 224 receives voice data from the audio source connected to I/O unit 210 via the data collection unit 226, and may transmit the voice data to the server (see e.g. 106 in FIG. 1). In response, the server may operate the method as described in FIG. 5 to generate a fertility indicator prediction or an ovulation status prediction for the subject, and may respond with these predictions to the user device. The voice sample data may be stored in the voice sample database 228 along with the prediction data. Prediction unit 224 may determine predictive messages based on the generated predictions. The predictive messages may be displayed to a user of the mobile device 200 using display 204. The predictive messages may include a fertility indicator or an ovulation status prediction.

In an alternate embodiment, the prediction unit 224 of the mobile device 200 may include at least one predetermined voice feature, and may operate the method as described in FIG. 5 to generate a fertility status prediction or ovulation status prediction for the subject or user of the mobile device. In this alternate unit, the voice sample data may be stored in the voice sample database 228 along with the prediction data.

The data collection unit 226 receives voice sample data from an audio source connected to the I/O unit 210.

In one or more embodiments, the data collection unit 226 receives onboarding data from the subject. The data collection unit 226 may receive and store the onboarding information in the fertility indicator database 230. The data collection unit 226 may receive the onboarding data and may transmit it to a server. The data collection unit 226 may supplement the onboarding data that is received from the user or subject with mobile device data and mobile device metadata.

The voice sample database 228 may be a database for storing voice samples received by the user device 200. The voice sample database 230 may receive the data from the data collection unit 226.

The fertility indicator database 430 may be a database for storing onboarding data, predicted fertility indicators, and predicted ovulation statuses.

FIG. 2B shows a server diagram showing detail of the server 106 in FIG. 1. The server 250 includes one or more of a communication unit 252, a display 254, a processor unit 256, a memory unit 258, I/O unit 260, a user interface engine 264, and a power unit 266.

The communication unit 252 can include wired or wireless connection capabilities. The communication unit 252 can include a radio that communicates using standards such as IEEE 802.11a, 802.11b, 802.11g, or 802.11n. The communication unit 252 can be used by the server 250 to communicate with other devices or computers.

Communication unit 252 may communicate with a network, such as networks 104 (see FIG. 1).

The display 254 may be an LED or LCD based display, and may be a touch sensitive user input device that supports gestures.

The processor unit 256 controls the operation of the server 250. The processor unit 256 can be any suitable processor, controller or digital signal processor that can provide sufficient processing power depending on the configuration, purposes and requirements of the server 250 as is known by those skilled in the art. For example, the processor unit 256 may be a high performance general processor. In alternative embodiments, the processor unit 256 can include more than one processor with each processor being configured to perform different dedicated tasks. The processor unit 256 may include a standard processor, such as an Intel® processor or an AMD® processor.

The processor unit 256 can also execute a user interface (UI) engine 264 that is used to generate various UIs for delivery via a web application provided by the Web/API Unit 282, some examples of which are shown and described herein, such as interfaces shown in FIG. 4A-4G.

The memory unit 258 comprises software code for implementing an operating system 270, programs 272, prediction unit 274, voice sample database 278, fertility indicator database 280, and Web/API Unit 282.

The memory unit 258 can include RAM, ROM, one or more hard drives, one or more flash drives or some other suitable data storage elements such as disk drives, etc. The memory unit 258 is used to store an operating system 270 and programs 272 as is commonly known by those skilled in the art.

The I/O unit 260 can include at least one of a mouse, a keyboard, a touch screen, a thumbwheel, a track-pad, a track-ball, a card-reader, an audio source, a microphone, voice recognition software and the like again depending on the particular implementation of the server 250. In some cases, some of these components can be integrated with one another.

The user interface engine 264 is configured to generate interfaces for users to configure fertility indicator predictions and ovulation status predictions, view fertility indicator predictions and ovulation status predictions, view voice sample data, etc. The various interfaces generated by the user interface engine 264 may be transmitted to a user device by virtue of the Web/API Unit 282 and the communication unit 252.

The power unit 266 can be any suitable power source that provides power to the server 250 such as a power adaptor or a rechargeable battery pack depending on the implementation of the server 250 as is known by those skilled in the art.

The operating system 270 may provide various basic operational processes for the server 250. For example, the operating system 270 may be a server operating system such as Ubuntu® Linux, Microsoft® Windows Server® operating system, or another operating system.

The programs 272 include various user programs. They may include several hosted applications delivering services to users over the network, for example, period tracking applications, fertility tracking applications, and the like.

In one or more embodiments, the programs 272 may provide a public health platform that is web-based, or client server based via a Web/API Unit 282 that provides fertility information for a set of subjects. This may include a patient population of a medical professional who is a user of the public health platform. For example, the medical professional may be able to receive a view into fertility indicator predictions or an ovulation status predictions for their patients to provide healthcare to the subjects.

The prediction unit 274 receives voice data from a user device over a network at Web/API Unit 282, and may operate the method as described in FIG. 5 to generate a fertility indicator prediction or an ovulation status prediction for the subject. The server may respond with the fertility indicator prediction or an ovulation status prediction to the user device via a message from the Web/API Unit 282. The voice sample data may be stored in the voice sample database 278 along with the prediction data. Prediction unit 274 may determine predictive messages based on the at least one predetermined voice feature and the voice sample data.

The voice sample database 278 may be a database for storing voice samples received from the one or more user devices via Web/API Unit 282. The voice sample database 278 may include voice samples from a broad population of subjects interacting with user devices. The voice samples in voice sample database 278 may be referenced by a subject identifier that corresponds to an entry in the fertility indicator database 280. The subject identifier may be a de-identified number that provides a measure of privacy and security for the subject's information in the event that it is ever inadvertently disclosed, for example as the result of a hacking attempt. The voice sample database 278 may include voice samples for a population of subjects, including more than 10,000, more than 100,000 or more than a million subjects. The voice sample database 278 may include voice samples from many different audio sources, including passive sensor devices, user devices, smart speakers, smart watches, game systems, etc.

The fertility indicator database 280 may be a database for storing fertility indicator prediction or an ovulation status predictions generated by the prediction unit 274. The fertility indicator prediction or an ovulation status prediction database 280 may be referenced by a subject identifier that corresponds to an entry in the database 280. The subject identifier may be a de-identified number that provides a measure of privacy and security for the subject's information in the event that it is ever inadvertently disclosed, for example as the result of a hacking attempt. The fertility indicator prediction or an ovulation status prediction database 280 may include fertility indicator prediction or an ovulation status prediction database corresponding to voice samples for a population of subjects, including more than 1,000, more than 10,000 or more than 100,000 subjects.

The Web/API Unit 282 may be a web-based application or Application Programming Interface (API) such as a REST (REpresentational State Transfer) API. The API may communicate in a format such as XML, JSON, or other interchange format.

The Web/API Unit 282 may receive a fertility indicator prediction or an ovulation status prediction request including a voice sample, may apply methods herein to determine a fertility indicator prediction or an ovulation status prediction, and then may provide the prediction in a fertility indicator prediction or an ovulation status prediction database response. The voice sample, values determined from the voice sample, and other metadata about the voice sample may be stored after receipt of a fertility indicator prediction or an ovulation status prediction request in voice sample database 278. The predicted fertility indicator or an ovulation status level may be associated with the voice sample database entry, and stored in the fertility indicator database 280.

Referring next to FIG. 3, there is shown a menstrual cycle diagram 300 of a subject in accordance with one or more embodiments.

The human menstrual cycle may be described in different phase categories 302, as a number corresponding to the number of days relative to ovulation 304, as a percentage chance of conception 306 if the subject were to have sexual intercourse or artificial insemination, and as a fertility category 308.

The menstrual cycle is broken up into three phases 302: the follicular phase, the luteal phase, and menstruation. The follicular phase begins with the first day of menstruation. In a non-pregnant individual, the uterus sheds its lining. Menstruation occurs on average for 3-7 days. After menstruation, estrogen levels begin to rise. When estrogen levels are sufficiently high, individuals produce follicle stimulating hormone (FSH) and luteinizing hormone (LH), both of which prepare the egg for release from the ovary. LH negatively inhibits estrogen production, so estrogen levels begin to decline. When LH peaks, ovulation occurs, indicating the end of the follicular phase. The luteal phase begins with ovulation. After ovulation, progesterone levels increase. LH levels begin to decrease, and estrogen levels increase again. After (average) 12.4 days, both hormones decrease and menstruation begins, indicating the end of the luteal phase and the beginning of the follicular phase.

The menstrual diagram 300 is shown along a time axis corresponding to the days relative to ovulation 304. Each of the phase 302, chance of conception 306, fertility category 308, and fertile window 310 may be described based on the number of days relative to ovulation 304.

The fertile window 310 may refer to the number of days relative to the subject's ovulation 304 where the subject may become pregnant as a result of sexual intercourse or artificial insemination.

The fertile window 310 including a high or very high likelihood of conception may be the 5 days prior to ovulation and the day after ovulation, and indicates the window in which an individual may become pregnant. The 5 days prior to ovulation may be derived from the lifespan of sperm, and the one day after may be derived from the lifespan of the egg.

The chance of conception 306 may be a predetermined statistical correlation corresponding to a percentage of <1% outside of the fertile window 304, and predetermined statistical correlations corresponding to the other days as shown.

The fertility category 308 corresponds to a “low”, “medium”, “high” and “very high” classifications as shown.

Referring next to FIGS. 4A, 4B, 4C, 4D, 4E, 4F, and 4G together, there are example user interfaces 400, 410, 420, 430, 440, 450, and 460 respectively showing a subject collecting a voice sample and receiving a fertility indicator prediction or an ovulation status prediction.

At the first execution of the application shown in FIGS. 4A, 4B, 4C, 4D, 4E, 4F, and 4G, the user may be required to enter onboarding information to configure the application. The onboarding may be performed the first time the user opens the application, or subsequently as requested by the user.

The onboarding information can include Age information, the First day of the users' menses, a preferred time of the day for voice sample collection, and a response to the question “Are you currently on birth control”. If the user answers yes, the system may indicate that the application is inappropriate for use for conception planning.

In one embodiment, the onboarding may further include recording or receiving a consent voice sample from the user. This may include, for example, a static prompt that the user may speak such as “I consent to collecting daily voice samples for the purposes of conception planning including identifying ovulation and fertility” or the like. The onboarding may determine that the consent voice sample may contain the correct phrase, for example, using a text-to-speech algorithm as known.

This verbal sample including consent may then be used by the software application at each daily voice sample collection to authenticate the collected voice sample against the acoustic fingerprint from the consent voice sample collected during onboarding. In the case where the authentication fails, the software application may stop and cease predictions based on the collected voice sample. This may increase the privacy of the fertility prediction and ovulation status prediction system by eliminating non-authenticated predictions and non-consensual data collection by the system by checking that the same voice is being used for analysis and predictions.

At interface 400, there is a user interface shown to a user at a user device 402 who desires to receive a fertility indicator or an ovulation status prediction. To initiate the prediction, the user is prompted to begin the fertility check by selecting a start button 406. Once start is selected, the audio input of the user device begins recording the voice sample into memory of the user device 402.

In an alternate embodiment, the user may receive a notification on the user device 402 to initiate the voice sampling, and by selecting the notification may be presented with interface 400 to initiate the collection. The notification to the user to initiate the voice sampling may be determined based on the time of day.

In response to the user selecting the start button, a variable prompt interface 410 is shown, prompting the user to read the prompt 414. The prompt may be a variable prompt 414 as shown, and may change subject to subject, or for each voice sample that is recorded. During the voice sample collection, the user interface 410 may show a voice sample waveform 416 on the display.

Alternatively, a static prompt to user interface 420 may instead be shown to a subject and the prompt 424 may be static. Each subject may speak the same prompt out loud for every voice sample. During the voice sample collection, the user interface 420 may show a voice sample waveform 426 on the display.

In one or more embodiments, in order to provide an added measure of privacy, the onboarding information may intentionally avoid collecting personally identifying information from the user, such as name, birthdate, phone number, etc.

In the case of either the interface 410 or the interface 420, the interface may further ask the user one or more questions at the time the user reads the prompt 414 or 424. For example, the interface may ask the user “Are you menstruating today” and may record a text based answer. Alternatively, a voice prompt may follow the user reading the prompt 414 or 424.

In response to completing the voice prompt (either static or variable), a fertility indicator or an ovulation status prediction 434 may be made in a fertility indicator or an ovulation status prediction interface 430. The fertility indicator or an ovulation status prediction 434 may be a categorical prediction (see e.g. fertility category 308 in FIG. 3), i.e. ‘Low’, ‘Medium’, ‘High’, and ‘Very High“. The fertility indicator or an ovulation status prediction 434 may be a categorical prediction (see e.g. phase category 302 in FIG. 3) such as ‘menstruation’, ‘follicular’ and ‘luteal’. The fertility indicator or an ovulation status prediction 434 may be an indicator of days relative to ovulation (see e.g. days relative to ovulation 304 in FIG. 3). The fertility indicator or an ovulation status prediction 434 may be a numerical prediction (see e.g. chance of conception 306 in FIG. 3) including a percentage. As described herein, the fertility indicator or an ovulation status prediction 434 may be for a plurality of predictions including two or more of the above noted categorical, indicator of days, or numerical prediction. The prediction may be generated by a server, or may be generated by the user device itself.

At user interface 440, an alternate user interface is shown including a prediction that is fertility indicator category of ‘Luteal’ 442.

At user interface 450, another alternate user interface is shown including a prediction that is a fertility indicator category of ‘High’ 452. The interface 450 may further include a numerical fertility prediction 454 that indicates to the user that they are “Fertile for the next 3 days”. The interface 450 may further include a timeline interface 456 including an indicator 458 of the subject's position along the timeline interface. The interface 450 may be output including the fertility level indicator by displaying the timeline interface on a display device of a user device.

The timeline interface 456 is shown in further detail in interface portion 460. The timeline interface 456 includes a menstruation windows 462a and 462b, a first non-fertile window 464a, a fertile window 466, a second non-fertile window 464b, and an indicator 458 of the subject's position along the timeline interface.

Referring next to FIG. 5, there is shown a computer-implemented method diagram 500 for predicting a fertility indicator or an ovulation status level. The method of FIG. 5 may operate at a user device such as a mobile device or laptop, a server, or a dedicated device associated with a user device.

The predicted fertility indicator or an ovulation status level may be represented as a category, a numerical value, a text description, or another type of representation describing the subject's predicted fertility indicator or an ovulation status level. The predictions may include those found in FIG. 3. The predictions may be output to the user, for example, the user device may output the predictions using a user interface such as the ones in FIGS. 4A-4G.

At 502, a voice sample from the subject is received at a processor in communication with a memory. The voice sample may be based on a user prompt that may include a sentence for the subject to vocalize. The sentence may be predetermined, randomized, or partially predetermined and partially randomized. The voice sample may be of different lengths, but in a preferred embodiment may be a single sentence. The voice sample that is recorded may be a voice command issued to a user device, such as one given to Apple® Siri®, Ok Google®, or Amazon® Alexa®. The voice sample may be encoded in various different digital audio file formats.

At 504, extracting at least one voice feature value from the voice sample for at least one predetermined voice feature at the processor. The at least one voice feature may be extracted using a software method to generate one or more signals based on the voice sample. For example, OpenSMILE may be used to select one or more feature signals to be extracted. For example, OpenSMILE may extract feature signals from the voice sample. In addition, the extraction using OpenSMILE, for example, the feature signals may be processed using one or more audio processing libraries or algorithms. For example, Firestore and Pandas libraries may be used for data downloading and dataframe manipulation of the extracted features signals from OpenSMILE.

The at least one predetermined voice feature may include fundamental frequency features such as the OpenSMILE features
F0semitoneFrom27.5 Hz_sma3nz_amean,
F0semitoneFrom27.5 Hz_sma3nz_stddevNorm (herein referred to as F0stdv),
F0semitoneFrom27.5 Hz_sma3nz_percentile20.0, and/or
F0semitoneFrom27.5 Hz_sma3nz_pctlrange0-2.

The at least one predetermined voice feature may include spectral flux features such as the OpenSMILE features spectralFlux_sma3_amean and/or spectralFluxV_sma3nz_amean.

The at least one predetermined voice feature may include jitter features such as jitterLocal_sma3nz_stddevNorm.

The at least one predetermined voice feature may include harmonic noise ratio features such as HNRdBACF_sma3nz_amean.

The at least one predetermined voice feature may include alpha ratio features such as OpenSMILE features alphaRatioV_sma3nz_stddevNorm and/or alphaRatioUV_sma3nz_amean.

At 506, determining an ovulation status for the subject based on the at least one voice feature value at the processor. The determining the ovulation status may include an analysis of the first derivative of the standard deviation of the fundamental frequency. For example, when the derivative becomes negative, an ovulation status prediction may be made corresponding to the ovulation/fertile window beginning.

Optionally, the determining the ovulation status may be performed using a pre-trained machine-learning model.

Optionally, a decision tree model may be used in order to determine an ovulation status. The decision tree may include one or more confidence values.

At 508, outputting (i) a fertility level indicator for the subject based on the ovulation status, and/or (ii) an ovulation status indicator for the subject based on the ovulation status, at an output device. The fertility indicator and the ovulation status indicator may be those shown in FIG. 3 and FIGS. 4A-4G.

Optionally, the fertility level indicator may be determined by adding five days to the first day the first derivative of fundamental frequency become negative. This prediction thus may provide a user with information that may assist them in becoming pregnant or avoiding pregnancy.

Optionally, the fertility level indicator for the subject may include a historical fertility indicator for the subject, including wherein the historical fertility indicator is provided over a single menstrual cycle of the subject.

Optionally, the fertility level indicator for the subject may be a category comprising fertile or not fertile.

Optionally, the fertility level indicator for the subject may be a category comprising: menstruating, follicular, or luteal.

Optionally, the fertility level indicator for the subject may be a category comprising: a low category, a medium category, and a high category.

Optionally, the low category, the medium category, and the high category each include predetermined thresholds.

Optionally, the ovulation status indicator may include an indicator of ovulation based on a transition from the follicular category to the luteal category.

Optionally, the fertility level indicator may include a percentage.

Optionally, the at least one predetermined voice feature may be at least one selected from a group of a fundamental frequency (F0) feature, a spectral flux feature, a jitter feature, a harmonic to noise ratio feature, a shimmer feature, and an alpha ratio feature.

Optionally, the at least one predetermined voice feature may include a fundamental frequency standard deviation feature, and wherein the determining, at the processor, the ovulation status for the subject may include: determining, at the processor, the at least one voice feature value comprising a mean fundamental frequency standard deviation of the voice sample and a deviation of the fundamental frequency standard deviation of the voice sample from the mean fundamental frequency standard deviation of the voice sample; and when the deviation is greater than a predetermined threshold, determine an occurrence of ovulation.

Optionally, the predetermined threshold may be 20%.

Optionally, the determining, at the processor, the ovulation status for the subject may include: determining, at the processor, the at least one voice feature value comprising a derivative of the fundamental frequency (F0) feature; and determining the ovulation status based on the derivative of the fundamental frequency (F0) feature.

Optionally, the at least one predetermined voice feature may include a non-patient specific feature.

Optionally, the ovulation status may be determined based on a negative derivative of the fundamental frequency (F0).

Optionally, the at least one predetermined voice feature may be a shimmer mean feature and wherein the determining the ovulation status for the subject may include: determining the at least one voice feature value comprising at least one local maximum shimmer mean feature value of the voice sample, and determining the ovulation status based on the at least one local maximum shimmer mean feature value.

Optionally, the ovulation status may be determined based a decision tree, the decision tree using the at least one predetermined voice feature of the voice sample to determine the ovulation status.

Optionally, the method may further comprise: receiving, at the processor from a user input device, onboarding information comprising an age of the subject, a first day of a menstrual cycle of the subject, a menstrual status of the subject, and optionally a birth control status of the subject; and wherein the method further comprises determining, at the processor, the ovulation status for the subject based on at least one selected from a group of the age of the subject, the first day of a menstrual cycle of the subject, the menstrual status of the subject, and optionally on the birth control status of the subject.

Optionally, the onboarding information may include an initial voice sample from the subject, the initial voice sample for indicating the subject's consent to the determining and outputting the ovulation status.

Optionally, the method may further comprise: authenticating the subject by comparing the voice sample to the initial voice sample prior to performing the determining and outputting of the ovulation status.

Optionally, the method may further comprise: deleting the voice sample.

Optionally, the fertility level indicator for the subject may include a timeline interface for the subject; and wherein the outputting the fertility level indicator comprises displaying the timeline interface on a display device.

Optionally, the timeline interface comprises a menstruation window, a first non-fertile window, a fertile window, a second non-fertile window, and an indicator of the subject's position along the timeline interface.

Optionally, the method may further comprise: receiving, at an audio input device, the voice sample; wherein the voice sample may be collected contemporaneously from the subject's speech.

Optionally, the method may further comprise: receiving, at an audio input device, the voice sample; and wherein the voice sample may comprise a prompt vocalized by the subject, optionally wherein the predetermined phrase comprises a date or a time.

Optionally, the method may further comprise: displaying, at the display device, a reminder notification to the subject to collect the voice sample.

Optionally, the reminder notification may be displayed to the subject at a predetermined time of the day.

Optionally, the reminder notification may comprise the predetermined phrase.

Optionally, the method may further comprise: providing, at a user device, a conception application for assisting the subject to become pregnant; wherein the voice sample is obtained at the user device using the conception application.

Optionally, the method may further comprise: generating, at the user device, a conception notification associated with the conception application, the conception notification comprising the fertility level indicator.

Optionally, the conception notification may be generated based on a percentage value of the fertility level indicator.

Optionally, the method may further comprise: providing, at a user device, a contraception application for assisting the subject to avoid becoming pregnant.

Optionally, the method may further comprise: generating, at the user device, a contraception notification associated with the contraception application, the contraception notification comprising the fertility level indicator.

Optionally, the contraception notification may be generated based on a percentage value of the fertility level indicator.

Optionally, the user device may be used by the subject.

Optionally, the user device may be used by a clinician.

Referring next to FIG. 6, there is shown a computer implemented method diagram 600 in accordance with one or more embodiments. During onboarding, as described above in reference to FIGS. 4A-4G, a user may be asked whether they are currently taking hormonal birth control medication. The use of birth control necessarily affects the predictions made in the method of FIG. 5. The determinations of FIG. 6 therefore may be used to improve the predictions of FIG. 5. This may be performed by combining the fertility indicator or ovulation status indicator with the determined hormonal birth control status to provide an improved prediction which includes whether or not the user is presently taking hormonal birth control. This may function as backup detection to the question provided during onboarding.

At 602, the average standard deviation of the fundamental frequency of the voice sample is determined and compared to a predetermined threshold. Alternatively, the average derivative of the standard deviation may be determined and compared with a predetermined threshold. If, for example, average standard deviation of the fundamental frequency is greater than 30 or if the average derivative of the standard deviation is greater than 400, then the method may automatically determine at 604 that the user is not using hormonal birth control, or may proceed to step 606.

At 604, the user may be automatically identified as not taking hormonal birth control.

At 606, the mean of the logarithm of the 1^{st} and 2^{nd} harmonics compared relatively to fundamental frequency is determined. This mean may be compared to a predetermined threshold. For example, if the mean is greater than 5, then the user may be automatically identified at 608 as taking hormonal birth control. If the mean is less than 5 then the user may automatically be identified as not taking hormonal birth control.

While the above description provides examples of one or more processes or systems, or computer program products, it will be appreciated that other processes or systems, or computer program products may be within the scope of the accompanying claims.

To the extent any amendments, characterizations, or other assertions previously made (in this or in any related patent applications or patents, including any parent, sibling, or child) with respect to any art, prior or otherwise, could be construed as a disclaimer of any subject matter supported by the present disclosure of this application, Applicant hereby rescinds and retracts such disclaimer. Applicant also respectfully submits that any prior art previously considered in any related patent applications or patents, including any parent, sibling, or child, may need to be re-visited.

### EXAMPLES

### Example 1: Biomarker Potential of Real-World Voice Signals to Predict Ovulation Status Indicators or Fertility Indicators

A study was performed to investigate whether ovulation or hormone concentrations could be detected from the voice and ovulation status indicators or fertility indicators could be predicted based on an associated bio-marker.

### Methods

OpenSMILE 88 feature signal extraction was used, as well as standard Firestore and Pandas libraries for data downloading and dataframe manipulation.

Each subject took their basal body temperature, an LH hormone test, and a voice recording every day for one menstrual cycle. Subjects were instructed to say the sentences: “Hello, how are you? Today is [current date] and the time is [current time]” for the voice recording. During menstruation, the LH T/C ratio was recorded as 0. Ovulation occurs approximately 24 hours after the LH peak, so the ovulation day was determined by calculating the day of the LH peak+1 day.

Subject's recordings were labeled and split into three classes: menstruation, follicular, luteal. A student's t-test was performed (2 tailed, unequal variance) with a significance value of p<0.05 between follicular vs luteal phase. Features with significant p value were incorporated for signal detection. Further analysis was performed comparing the fertile window (5 days preceding ovulation) and the ovulation day for all individuals (using Student's t-test and p<0.05).

Another analysis was conducted to separate individuals on hormonal birth control and naturally cycling women. Data from non-menstruating individuals on hormonal birth control was compared to the follicular and luteal phase of naturally cycling individuals. Mean values were compared using Student's t-test and p<0.05.

### Results

Statistical significance in the mean values for the follicular phase vs the luteal phase were observed in the following features.

Fundamental frequency: F0semitoneFrom27.5 Hz_sma3nz_amean,
F0semitoneFrom27.5 Hz_sma3nz_stddevNorm (herein referred to as F0stdv),
F0semitoneFrom27.5 Hz_sma3nz_percentile20.0,
F0semitoneFrom27.5 Hz_sma3nz_pctlrange0-2,
Spectral Flux: spectralFlux_sma3_amean, spectralFluxV_sma3nz_amean
Jitter: jitterLocal_sma3nz_stddevNorm
Harmonic Noise Ratio: HNRdBACF_sma3nz_amean
Alpha Ratio: alphaRatioV_sma3nz_stddevNorm, alphaRatioUV_sma3nz_amean

Referring to FIG. 7, there is shown a result diagram showing the day-to-day standard deviation of the fundamental frequency (F0). It was found that there is a sharp/significant drop in F0stdv 0-5 days prior to ovulation (0-4 days prior to LH surge) for all naturally cycling individuals (see e.g. ovulation indication 702 in FIG. 7). This is during the fertile window, which is useful in conception planning.

Challenges were noted using this signal because of a decrease in F0stdv occurring multiple times throughout the cycle. So, the meaningful F0stdv decrease must be extracted and separated from the other values. The example method for extraction is as follows: the first decrease starting the fifth day of data recording that has an absolute value greater than 20% of the mean F0stdv of the previous data will be the signal.

Referring to FIG. 8, another result diagram is shown including further analysis that was performed comparing the results of the fertile window to the day of ovulation. The shimmer mean value over the voice recording was statistically significant (p=0.018). When viewing the shimmer mean value across the time series in FIG. 8, the shimmer mean is a local maximum on the day of ovulation (feature shimmerLocaldB_sma3nz_amean). A time series of the average shimmer values can be seen below, with the red arrow indicating the day of ovulation (see e.g., reference 802 in FIG. 8).

### REFERENCES

[1] Shoup-Knox, Melanie L., et al. “Fertility-Dependent Acoustic Variation in Women's Voices Previously Shown to Affect Listener Physiology and Perception.” Evolutionary Psychology 17.2 (2019): 1474704919843103.
[2] Pavela Banai, Irena. “Voice in different phases of menstrual cycle among naturally cycling women and users of hormonal contraceptives.” PLoS One 12.8 (2017): e0183462.
[3] Fischer, Julia, et al. “Do women's voices provide cues of the likelihood of ovulation? The importance of sampling regime.” PloS one 6.9 (2011): e24490.
[4] Zamponi, Virginia, et al. “Effect of sex hormones on human voice physiology: from childhood to senescence.” Hormones 20.4 (2021): 691-696.

We claim:

1. A computer-implemented method for providing a fertility indicator or an ovulation status for a subject, the method comprising:
receiving, at a processor in communication with a memory, a voice sample from the subject;
extracting, at the processor, at least one voice feature value from the voice sample for at least one predetermined voice feature;
determining, at the processor, an ovulation status for the subject based on the at least one voice feature value; and
outputting, at an output device, (i) a fertility level indicator for the subject based on the ovulation status, and/or (ii) an ovulation status indicator for the subject based on the ovulation status.

2. The method of claim 1, wherein the fertility level indicator for the subject comprises a historical fertility indicator for the subject, optionally wherein the historical fertility indicator is provided over a single menstrual cycle of the subject.

3. The method of claim 2, wherein the fertility level indicator for the subject is a category comprising fertile or not fertile.

4. The method of claim 2, wherein the fertility level indicator for the subject is a category comprising: menstruating, follicular, or luteal; and wherein the ovulation status indicator comprises an indicator of ovulation based on a transition from the follicular category to the luteal category.

5. The method of claim 3, wherein the fertility level indicator for the subject is a category comprising: a low category, a medium category, and a high category and optionally wherein the low category, the medium category, and the high category each comprise predetermined thresholds.

6. The method of claim 3, wherein the fertility level indicator comprises a percentage; and wherein the at least one predetermined voice feature is at least one selected from a group of a fundamental frequency (F0) feature, a spectral flux feature, a jitter feature, a harmonic to noise ratio feature, a shimmer feature, and an alpha ratio feature.

7. The method of claim 6, wherein the at least one predetermined voice feature comprises a fundamental frequency standard deviation feature, and wherein the determining, at the processor, the ovulation status for the subject comprises:
determining, at the processor, the at least one voice feature value comprising a mean fundamental frequency standard deviation of the voice sample and a deviation of the fundamental frequency standard deviation of the voice sample from the mean fundamental frequency standard deviation of the voice sample;
and when the deviation is greater than a predetermined threshold, determine an occurrence of ovulation.

8. The method of claim 7, wherein the determining, at the processor, the ovulation status for the subject comprises:
determining, at the processor, the at least one voice feature value comprising a derivative of the fundamental frequency (F0) feature; and
determining the ovulation status based on the derivative of the fundamental frequency (F0) feature, wherein the ovulation status is determined based on a negative derivative of the fundamental frequency (F0).

9. The method of claim 8, wherein the at least one predetermined voice feature is a shimmer mean feature and wherein the determining the ovulation status for the subject comprises:
determining the at least one voice feature value comprising at least one local maximum shimmer mean feature value of the voice sample, and
determining the ovulation status based on the at least one local maximum shimmer mean feature value; and
wherein the ovulation status is determined based a decision tree, the decision tree using the at least one predetermined voice feature of the voice sample to determine the ovulation status.

10. The method of claim 1 further comprising:
authenticating the subject by comparing the voice sample to the initial voice sample prior to performing the determining and outputting of the ovulation status.

11. The method of claim 10, wherein the fertility level indicator for the subject comprises a timeline interface for the subject; and wherein the outputting the fertility level indicator comprises displaying the timeline interface on a display device; and wherein the timeline interface comprises a menstruation window, a first non-fertile window, a fertile window, a second non-fertile window, and an indicator of the subject's position along the timeline interface.

12. The method of claim 1, further comprising:
providing, at a user device, a conception application for assisting the subject to become pregnant;
generating, at the user device, a conception notification associated with the conception application, the conception notification comprising the fertility level indicator;
wherein the conception notification is generated based on a percentage value of the fertility level indicator;
wherein the voice sample is obtained at the user device using the conception application.

13. The method of claim 1, further comprising:
providing, at a user device, a contraception application for assisting the subject to avoid becoming pregnant;
generating, at the user device, a contraception notification associated with the contraception application, the contraception notification comprising the fertility level indicator; and
wherein the contraception notification is generated based on a percentage value of the fertility level indicator.

14. A system for determining a fertility level for a subject, the system comprising:
a memory;
a processor in communication with the memory, the processor configured to:
receive a voice sample from the subject;
extract at least one voice feature value from the voice sample for at least one predetermined voice feature;
determine an ovulation status for the subject based on the at least one voice feature value; and
output, at an output device, (i) a fertility level indicator for the subject based on the ovulation status, and/or (ii) an ovulation status indicator for the subject based on the ovulation status.

15. The system of claim 14, wherein the fertility level indicator for the subject comprises a historical fertility indicator for the subject, optionally wherein the historical fertility indicator is provided over a single menstrual cycle of the subject.

16. The system of claim 15, wherein the fertility level indicator for the subject is a category comprising fertile or not fertile.

17. The system of claim 15, wherein the fertility level indicator for the subject is a category comprising: menstruating, follicular, or luteal; and wherein the ovulation status indicator comprises an indicator of ovulation based on a transition from the follicular category to the luteal category.

18. The system of claim 16, wherein the fertility level indicator for the subject is a category comprising: a low category, a medium category, and a high category and optionally wherein the low category, the medium category, and the high category each comprise predetermined thresholds.

19. The system of claim 16, wherein the fertility level indicator comprises a percentage; and wherein the at least one predetermined voice feature is at least one selected from a group of a fundamental frequency (F0) feature, a spectral flux feature, a jitter feature, a harmonic to noise ratio feature, a shimmer feature, and an alpha ratio feature.

20. The system of claim 19, wherein the at least one predetermined voice feature comprises a fundamental frequency standard deviation feature, and wherein the processor determines the ovulation status for the subject by:
determining the at least one voice feature value comprising a mean fundamental frequency standard deviation of the voice sample and a deviation of the fundamental frequency standard deviation of the voice sample from the mean fundamental frequency standard deviation of the voice sample;
and when the deviation is greater than a predetermined threshold, determine an occurrence of ovulation.

21. The system of claim 20, wherein the processor determines the ovulation status for the subject by:
determining the at least one voice feature value comprising a derivative of the fundamental frequency (F0) feature; and
determining the ovulation status based on the derivative of the fundamental frequency (F0) feature, wherein the ovulation status is determined based on a negative derivative of the fundamental frequency (F0).

22. The system of claim 21, wherein the at least one predetermined voice feature is a shimmer mean feature and wherein the determining the ovulation status for the subject comprises:
determining the at least one voice feature value comprising at least one local maximum shimmer mean feature value of the voice sample, and
determining the ovulation status based on the at least one local maximum shimmer mean feature value; and
wherein the ovulation status is determined based a decision tree, the decision tree using the at least one predetermined voice feature of the voice sample to determine the ovulation status.

23. The system of claim 14 wherein the processor is further configured to:
authenticate the subject by comparing the voice sample to the initial voice sample prior to performing the determining and outputting of the ovulation status.

24. The system of claim 23, wherein the fertility level indicator for the subject comprises a timeline interface for the subject; and wherein the outputting the fertility level indicator comprises displaying the timeline interface on a display device; and wherein the timeline interface comprises a menstruation window, a first non-fertile window, a fertile window, a second non-fertile window, and an indicator of the subject's position along the timeline interface.

25. The system of claim 14, further comprising:
a conception application provided at a user device for assisting the subject to become pregnant, the conception application generating a conception notification at the user device, the conception notification comprising the fertility level indicator;
wherein the conception notification is generated based on a percentage value of the fertility level indicator;
wherein the voice sample is obtained at the user device using the conception application.

26. The system of claim 25, further comprising:
a contraception application provided at a user device for assisting the subject to avoid becoming pregnant, the contraception application generating a contraception notification at the user device, the contraception notification comprising the fertility level indicator;
the contraception application generating a contraception notification associated with the contraception application, the contraception notification comprising the fertility level indicator; and
wherein the contraception notification is generated based on a percentage value of the fertility level indicator.
